(19) United States
(12) Patent Application Publication
(10) Pub. No.: US 2024/0168711 A1
(43) Pub. Date: May 23, 2024

(54) OBJECT GROUP PACKING

(71) Applicants: Hewlett-Packard Development Company, L.P., Spring, TX (US); INSTITUTO ATLÂNTICO, Edson Queiroz, Fortaleza (BR)

(72) Inventors: Juan Carlos CATANA SALAZAR, Guadalajara JAL (MX); Alyne GOMES SOARES CANTAL, Porto Alegre, Rio Grande do Sul (BR); Andre Luiz LIMA PEDRO, Porto Alegre, Rio Grande do Sul (BR); Scott Alan WHITE, Boise, ID (US); Jun ZENG, Palo Alto, CA (US)

(21) Appl. No.: 18/283,568

(22) PCT Filed: Apr. 1, 2021
(86) PCT No.: PCT/US2021/025393

§ 371 Date: Sep. 22, 2023

(51) Int. Cl. G06F 7/08; B33Y 50/00; G16B 15/10; G16B 35/00; G16B 40/00

(52) U.S. Cl. CPC G06F 7/08; G16B 15/10; G16B 35/00; G16B 40/00; B33Y 50/00

(57) ABSTRACT

Examples of methods for object group packings are described herein. In some examples, a method includes ordering objects of a chromosome into a first group and a second group in accordance with a first priority and a second priority. In some examples, the method includes determining, using a genetic procedure in accordance with the first priority and the second priority, a first arrangement within the first group and a second arrangement within the second group to produce a packing in a volume.

### BACKGROUND

Three-dimensional (3D) solid objects may be produced from a digital model using manufacturing. Additive manufacturing is an example of manufacturing. Additive manufacturing may be used in rapid prototyping, mold generation, mold master generation, and short-run manufacturing. Additive manufacturing involves the application of successive layers of build material. In some additive manufacturing techniques, the build material may be cured or fused. Other machining processes remove material to create the final object.

### BRIEF DESCRIPTION OF THE DRAWINGS

FIG. 1 is a flow diagram illustrating an example of a method for object group packing;

FIG. 2 is a block diagram of an example of an apparatus that may be used in object group packing;

FIG. 3 is a block diagram illustrating an example of a computer-readable medium for performing object group packing; and

FIG. 4 is a diagram illustrating an example of recursive genetic procedures in accordance with some examples of the techniques described herein.

### DETAILED DESCRIPTION

Manufacturing techniques (e.g., additive manufacturing, three-dimensional (3D) printing) may be used to manufacture 3D objects. Some examples of manufacturing (e.g., 3D printing, additive manufacturing, etc.) may utilize a packing. A packing is information that specifies a positioning of objects in a volume. A positioning is a position(s), location(s), and/or orientation(s), etc., of an object or objects. An “object” may refer to a geometrical representation or 3D shape. A volume is a 3D space.

A build volume is a volume that may be used for manufacturing. In some examples, a build volume may correspond to a physical space in which additive manufacturing may be performed. In some examples, a volume, build volume, or volume subset may be structured as a 3D space bounded by a predefined polyhedron (e.g., a set of vertices, edges, and faces) that specifies the packing space scope. For example, a cuboidal space may be defined by 8 vertices (e.g., V={v1=(0,0,0), v2=(0,20,0), v3=(20,0,0), v4=(20,20,0), v5=(0,0,80), v6=(0,20,80), v7=(20,0,80), v8=(20,20,80)}), 12 edges (e.g., E={e1=(v1,v2), e2=(v1,v3), e3=(v1,v5), e4=(v2,v4), e5=(v2,v6), e6=(v3,v4), e7=(v3,v7), e8=(v4,v8), e9=(v5,v6), e10=(v5,v7), e11=(v6,v8), e12=(v7,v8)}), and 6 faces (e.g., F={f1={e1,e7,e10,e3}, f2={e6,e8,e12,e7}, f3={e8,e11,e5,e4}, f4={e2,e5,e9,e3}, f5={e1,e6,e4,e2}, f6={e10,e12,e11,e9}}). Different vertices, edges, and/or faces (and/or quantities thereof) may be utilized in some examples. In some examples, a build volume (e.g., packing space) may be a cuboidal space with x, y, and z dimensions. For example, a build volume may be a cuboid with dimensions of 284 millimeters (mm) in the x axis by 380 mm in the y axis by 380 mm in the z axis.

In some examples, object packing may be based on an objective or objectives. An example of an objective is packing density (e.g., increasing or maximizing packing density). Object packing questions may be computationally intensive to solve, being nondeterministic polynomial- (NP-) complete. For example, computational complexity may grow exponentially in NP-complete questions. For instance, the size of the solution space (e.g., quantity of possible packings) for placement of objects (without rotation, for example) in a relatively small batch of 20 objects, is beyond the order of 1×10¹⁸.

Some of the techniques described herein may be utilized to prioritize an object or objects in packing objects into a volume. It some cases, it may be helpful to prioritize object packing for manufacturing. For instance, when selecting objects (from a pool of objects, for example) for packing into a build volume for batch production, an object or group of objects may be given priority for entrance into the build volume, for packing in the build volume, and/or for manufacturing. In some examples, an object or objects with lower priority may bear a risk of being left out of the current build volume and/or waiting for a subsequent build volume. A priority is a rank, level in a hierarchy, and/or position in an order.

In some examples, objects with a same or similar priority may be packed near each other in the volume. For instance, objects with a same or similar priority may be packed at a same or similar z-height in the volume. Packing objects with a similar priority at a similar z-height may be helpful for some types of additive manufacturing technologies (e.g., multi-jet fusion (MJF)), where manufactured object quality may depend on the object's packing location in the z dimension. In some examples, objects that are assigned the same priority may have similar quality, may be manufactured in similar time frames, and/or may be shipped out in similar time frames.

Throughout the drawings, identical reference numbers may or may not designate similar or identical elements. Similar numbers may or may not indicate similar elements. When an element is referred to without a reference number, this may refer to the element generally, with or without limitation to any particular drawing or figure. The drawings are not necessarily to scale, and the size of some parts may be exaggerated to more clearly illustrate the example shown. Moreover, the drawings provide examples in accordance with the description. However, the description is not limited to the examples provided in the drawings.

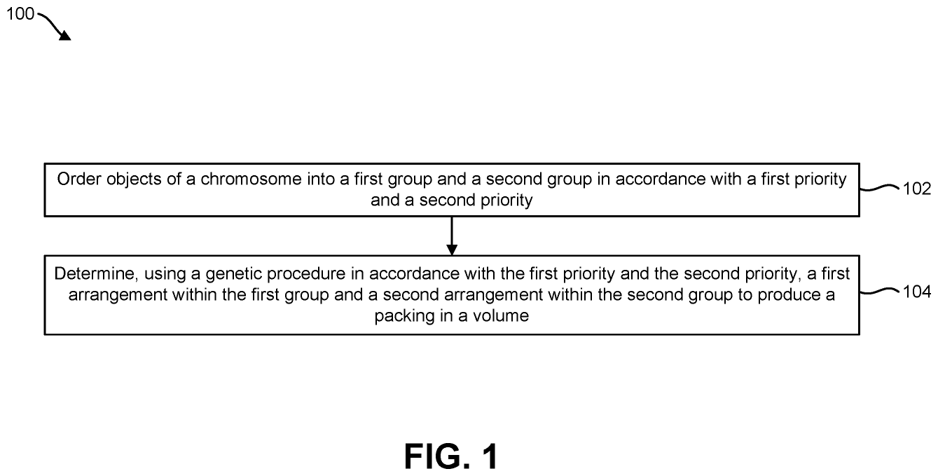

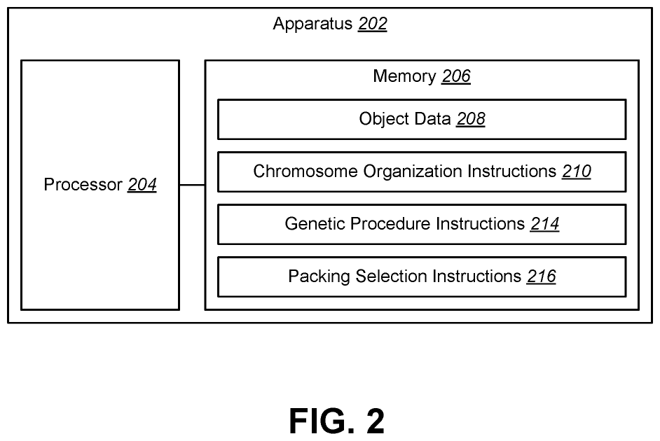

FIG. 1 is a flow diagram illustrating an example of a method 100 for object group packing. The method 100 and/or a method 100 element or elements may be performed by an apparatus (e.g., electronic device). For example, the method 100 may be performed by the apparatus 202 described in relation to FIG. 2.

An apparatus may order 102 objects of a chromosome into a first group and a second group in accordance with a first priority and a second priority. A chromosome is data. For example, a chromosome may include data representing a potential solution for a question that the genetic procedure is to address. A chromosome may include and/or represent a set of objects. In some examples, a chromosome may include data indicating a set of object identifiers and poses. An object identifier is information (e.g., number, floating point number, integer, string, character(s), name, etc.) that identifies an object. A pose is information indicating a location and/or orientation of an object. For instance, a pose may indicate a location (e.g., translation) of an object in a build volume and/or an orientation (e.g., rotation(s) in a dimension or dimensions) of an object in a build volume. In some examples, a pose may be expressed as a number or numbers (e.g., floating point numbers, integers, etc.), vector(s), matrix or matrices, quaternion(s), etc. In some examples, a chromosome may include an object identifier with a first axis rotation (e.g., x), a second axis rotation (e.g., y), and a third axis rotation (e.g., z) for each of a set of objects. In some examples, a gene of a chromosome may represent an object. In some examples, a chromosome may include an object identifier with an orientation (and without location, for instance) for each of a set of objects. In some examples, each gene of a chromosome may include an object identifier and/or pose (e.g., orientation and/or location).

In some examples, a chromosome may include data for each object in an order or sequence. In some examples, the order or sequence of a chromosome may correspond to a packing order in the volume (e.g., build volume). For instance, the apparatus may place the objects of a chromosome in the volume (e.g., build volume) in the order or sequence indicated in the chromosome. In some cases, consecutive objects in a chromosome may be placed adjacently in the volume if volume dimensions allow, to an extent that volume dimensions allow, and/or to an extent that previously-placed object(s) allow. In some examples, the order or sequence may establish a queue. For instance, the order or sequence may indicate an order that objects may be introduced into a build volume. In some examples, a chromosome may include an object identifier and rotations for respective objects from object 1 to object n. For instance, a chromosome may encode an object list (e.g., list of genes), where each element of the list indicates an identifier of an object and/or rotation angles corresponding to axes (e.g., in x, y, and z axes). In some examples, the order or sequence of a chromosome may be an order or sequence of object identifiers. In some examples, each chromosome may represent a potential packing described as a sequence of object identifiers with associated poses.

In some examples, a chromosome may encode a sequence of genes (objects with orientations, for instance) to be followed to enter the objects in the build volume. Each chromosome may lead to a different placing of objects in the build volume, and therefore different packings.

In some examples, partial gene ordering may be controlled in the chromosome to meet a priority criterion in the packing. A sequence of group priorities may be established. For instance, a higher priority group may be processed and/or packed first, and a lower priority group may be processed and/or packed later. A group is a collection of objects and/or representations (e.g., genes, object data, a chromosome subset, etc.) of objects.

In some examples, a chromosome may be ordered in accordance with group priorities. For example, first positions of the chromosome may be utilized for the objects in the first group, subsequent positions in the chromosome may be utilized for the objects in the second group, and so on. The chromosome may reflect the partial ordering given by the group priority.

In some examples, ordering 102 the objects may include sorting the objects. For instance, the apparatus may rank the objects and/or place objects in groups in accordance with a priority. An object or objects with a higher priority may be placed before an object or objects with a lower priority (e.g., higher priority objects may be packed and/or manufactured before lower priority objects). For example, a first group may have a first priority and a second group may have a second priority, where the first priority is higher than the second priority.

In some examples, ordering 102 the objects may include sorting the objects based on a sorting factor. A sorting factor is a characteristic, value, quantity, and/or indicator that indicates a sequence, ranking, and/or hierarchy. An object or objects may have an associated sorting factor and/or the apparatus may determine a sorting factor for the object or objects. Examples of sorting factors may include target region (e.g., a relative or absolute region in the build volume, relative or absolute z-height, etc.), geometrical feature(s) (e.g., feature complexity), timeline (e.g., manufacturing timeline, shipping timeline, etc.), object quantity, and/or priority input (e.g., received user input indicating a priority). In some examples, the groups may be computed automatically by classifying the objects into multiple groups and/or assigning priorities. In some examples, priority may be determined based on the sorting factor (e.g., geometrical features, dimensional properties, business priority, service level agreement, and/or another metric). Examples of sorting factors are given as follows.

A target region may be utilized to rank the objects where objects with a higher ranking are prioritized to a region (e.g., premium region, region more likely to produce higher quality, etc.) of the build volume. In some examples, a build volume may be graded and/or divided into multiple regions (e.g., price rate regions). Objects with more stringent manufacturing tolerances and/or at a higher price rate may be prioritized over objects with less stringent manufacturing tolerances and/or lower price rates. For example, an entity (e.g., user, business, etc.) may indicate more stringent manufacturing tolerance and/or may pay a higher rate for a region of the build volume (e.g., lower z-height(s)) that may produce increased quality relative to another region of a build volume (e.g., higher z-height(s)). The apparatus may associate a sorting factor (e.g., value, number, indicator, price class, manufacturing tolerance, etc.) that indicates a target region with each object. The apparatus may order the objects in accordance with the sorting factor (e.g., target region).

A geometrical feature or features (e.g., geometrical complexity) may be utilized to rank the objects where objects with a higher ranking are prioritized to a region (e.g., premium region, region more likely to produce higher quality, etc.) of the build volume. In some examples, objects with more complex geometry (e.g., smaller features, sharper features, etc.) and/or greater target geometrical precision may be prioritized over objects with less complex geometry and/or lesser target geometrical precision. For example, an entity (e.g., user, business, etc.) may indicate a target geometrical precision and/or an apparatus may determine a complexity measure (e.g., an indication of smaller features, narrower angles, and/or more structures, etc.) as a sorting factor of geometrical feature(s). The apparatus may associate the sorting factor that indicates a geometrical feature(s) with each object. The apparatus may order the objects in accordance with the sorting factor (e.g., geometrical feature(s)). For instance, objects with greater geometrical complexity may be placed in a region (e.g., lower z-height(s)) of the build volume that is more likely to provide greater manufacturing quality.

A timeline (e.g., manufacturing timeline and/or shipping timeline) may be utilized to rank the objects where objects with a higher ranking are prioritized for packing in the build volume (e.g., in a region with more reliable manufacturing, higher quality manufacturing, and/or lower z-height(s), etc.). In some examples, objects with a shorter timeline (e.g., earlier due date, shorter manufacturing period, etc.) may be prioritized over objects with a longer timeline (e.g., later due date, longer manufacturing period, etc.). For example, an entity (e.g., user, business, etc.) may indicate a timeline and/or an apparatus may determine a timeline (e.g., amount of time before a manufacturing and/or shipping deadline, etc.) as a sorting factor. The apparatus may associate the sorting factor that indicates a timeline with each object. The apparatus may order the objects in accordance with the sorting factor (e.g., timeline). For instance, objects with shorter timelines may be placed in a region (e.g., lower z-height(s)) of the build volume that is more likely to provide greater manufacturing quality. Objects with longer timelines may be placed in a region (e.g., higher z-height(s)) of the build volume that is less likely to provide manufacturing quality.

An object quantity (e.g., quantity of objects ordered) may be utilized to rank the objects, where objects in a larger set of objects are prioritized for packing in the build volume (to increase production speed and/or lower object cost). In some examples, objects in a greater quantity (e.g., larger order.) may be prioritized over objects with a lesser quantity (e.g., smaller order). For example, an entity (e.g., user, business, etc.) may indicate an object quantity and/or an apparatus may determine an object quantity (e.g., quantity of objects in an order) as a sorting factor. The apparatus may associate the sorting factor that indicates the object quantity with each object. The apparatus may order the objects in accordance with the sorting factor (e.g., object quantity).

A priority input (e.g., designated priority) may be utilized to rank the objects, where objects with a higher priority input are prioritized for packing in the build volume. For instance, the apparatus may receive a priority input from an input device indicating a priority for an object or objects. For example, the apparatus or another device may receive an input (e.g., keyboard input, touchscreen input, mouse input, wireless signal, and/or stylus input, etc.) that indicates the priority input. For example, an entity (e.g., user, business, etc.) may indicate a priority input and/or an apparatus may receive a priority input as a sorting factor. The apparatus may associate the sorting factor that indicates the priority input with each object. The apparatus may order the objects in accordance with the sorting factor (e.g., priority input).

In some examples, ordering 102 the objects may include placing the objects in the groups based on the sorting factor(s). For instance, objects having sorting factors that satisfy a threshold (e.g., target region threshold, geometrical feature threshold, timeline threshold, object quantity threshold, and/or priority input threshold) may be ordered into the first group, while other objects (e.g., objects having sorting factors that do not satisfy the threshold) may be ordered into the second group. For instance, objects with geometrical feature factors greater than or at least a geometrical feature threshold may be placed in the first group. In another example, objects with timeline factors less than or not more than a timeline threshold may be placed in the first group. The first group may be ordered before the second group in the chromosome. In some examples, the sorting factors may indicate the groups. For instance, priority inputs may indicate group numbers (e.g., “1,” “2,” “3,” etc.), which may be utilized to order the objects into corresponding groups. In some examples, other quantities of groups may be utilized. For instance, objects may be sorted into two, three, four, five, eight, ten, etc., groups. Groups may be ordered in priority order (e.g., from highest to lowest priority).

In some examples, ordering 102 the objects may be performed based on a combination of sorting factors. For instance, different types of sorting factors (e.g., target region, geometrical feature(s), timeline, object quantity, and/or priority input, etc.) may be combined and/or utilized to order 102 the objects. In some examples, a first sorting factor may be utilized to perform a first sorting, then a second sorting factor may be utilized to perform a second sorting (e.g., sub-sorting), etc. In some examples, different types of sorting factors may be mathematically combined (e.g., summed, used for a weighted sum, etc.) and the combined sorting factors may be utilized to order 102 the objects. In some examples, a geometrical feature or features such as triangle count for a shape mesh, number of points or vertices, volume, and/or surface size, etc., may be utilized for ordering (as a sorting factor or factors, for instance). In some examples, a geometrical feature or features such as compactness, shape descriptor, elongation, rectangularity, Fourier descriptor, and/or barycentric distance, etc., may be utilized for ordering (as a sorting factor or factors, for instance).

In some examples, a portion of a chromosome may correspond to the first group and a portion of the chromosome may correspond to the second group. For instance, a gene or genes of a chromosome may be included in the first group, and another gene or genes of the chromosome may indicate and/or represent the second group, etc. In some examples, a portion of a chromosome that indicates and/or represents the first group may be an initial portion of the chromosome, and/or a portion of a chromosome that indicates and/or represents the second group may be a last portion of the chromosome. For instance, the first group may be ordered for packing first in the sequence of the chromosome followed by the second group in some approaches. For instance, a chromosome may indicate a packing order of objects in a volume (e.g., build volume). A packing is an organization of objects. For instance, a chromosome may indicate an object attention queue or sequence, and a placement procedure may locate the objects in a build volume in the chromosome order.

In some examples, ordering 102 objects of a chromosome may include ordering the objects in multiple groups (e.g., two, three, four, five, etc., groups) according to priority. For instance, multiple groups may be ordered in a chromosome according to priority.

In some examples, the apparatus may perform a genetic procedure using a chromosome or chromosomes. A genetic procedure is a computational procedure. For example, a genetic procedure may be a metaheuristic procedure that includes evolution and/or selection mechanisms for determining a solution. In some examples, a genetic procedure may be a technique utilized in artificial intelligence. In some examples, the genetic procedure may include initializing a population, evaluation, selection, crossover, and/or mutation operations. In some examples, evaluation, selection, crossover, and/or mutation may be performed repeatedly (e.g., iteratively, recursively, etc.) until an end condition is met. In some examples, determining 104 a first arrangement and a second arrangement using a genetic procedure may be performed in initializing a population, crossover, and/or mutation operations. A population is a group or set of chromosomes.

In initializing a population, the apparatus may randomly initialize a set of chromosomes and/or portions of chromosomes. For example, the apparatus may utilize a random number generator to generate random poses for a set of objects (to be packed in a build volume, for instance). In some examples, the initialized set of chromosomes and/or portions of chromosomes may include sequences of object identifiers and poses. In some examples, the apparatus may initialize groups of objects of a chromosome or chromosomes. For instance, the apparatus may randomly initialize the first group of objects and the second group of objects. For instance, the apparatus may randomly initialize object ordering and/or poses within the first group, and may randomly initialize object ordering and/or poses within the second group. In some examples, initialization (e.g., random object ordering and/or random pose generation) may be performed within groups (e.g., intra-group initialization) without changing group membership (e.g., without moving an object out of a group and/or without adding an object to a group, etc.). In some examples, the apparatus may initialize a population of chromosomes, where different chromosomes may vary by intra-group ordering and/or pose. In some examples, different chromosomes may include the same group membership (e.g., object identifiers within groups may be the same).

In some examples, the priority groups may be processed as a single chromosome in a single genetic procedure instance. In some examples, the priority groups may preserve object membership throughout the genetic procedure. For instance, when a chromosome is created and/or when a mutation is performed, the object identifiers in the chromosome may be maintained and/or preserved inside the corresponding group scope. Mutations may modify object orientations and/or object sequence inside the priority groups. In some approaches, the genetic procedure may proceed (e.g., evaluation may be) based on global chromosome fitness. In some examples, processing a chromosome in a genetic procedure instance may increase a probability of convergence.

In performing evaluation, the apparatus may evaluate and/or rank the chromosomes according to a value or values. Examples of values include fitness scores. A fitness score is a measure, metric, or score that indicates a degree to which a chromosome (e.g., packing) satisfies an objective or objectives (e.g., increased packing density, decreased packing height, and/or decreased z-axis measure, etc.). For example, the fitness score may be evaluated for each chromosome (e.g., packing). Examples of fitness scores may include quantity of objects packed, packing density, and/or packing height (e.g., z-height). The quantity of objects packed is a quantity of objects that fit within the volume (e.g., build volume) for a chromosome. A quantity of objects packed may be determined for each chromosome.

In some examples, a value or values (e.g., fitness score, quantity of objects packed, packing density, and/or packing height, etc.) may be utilized to rank or order chromosomes. In some examples, a group of values (e.g., fitness score, quantity of objects packed, packing density, and/or packing height, etc.) may be assembled and/or combined to form a packing score that can be utilized to rank one packing over another packing. For instance, a packing score may be determined based on a function, combination, mapping, weighted sum, average, etc., of multiple values (e.g., fitness score, quantity of objects packed, packing density, and/or packing height, etc.). In some examples, chromosomes with a relatively greater packing density, a relatively greater quantity of objects packed, and/or a relatively lesser packing height may be ranked higher than other chromosomes.

In some examples, the value(s) (e.g., fitness score(s), packed quantity(ies), packing density(ies), and/or packing height(s), etc.) may be determined based on a placement procedure. An example of a placement procedure is a bottom-front-left placement procedure. For instance, the objects of a chromosome may be placed in the volume (e.g., build volume) in accordance with the placement procedure to produce a packing. The value(s) (e.g., fitness score(s), packed quantity(ies), packing density(ies), and/or packing height(s), etc.) may be determined (e.g., calculated, computed, etc.) based on the packing.

In some examples, a bottom-front-left placement procedure may utilize the object sequence of a chromosome to enter each object in sequence (e.g., one after another in the given order). The bottom-front-left placement procedure (e.g., heuristic) may enter an object from a top-back-right corner of a build volume (or volume subset), and move (e.g., incrementally move, continuously move, etc.) the object downward (e.g., along a z axis) until contacting with (or colliding with or coming within a distance from) another object or contacting with (or colliding with or coming within a distance from) a build volume (or volume subset) frontier (e.g., edge, boundary, face, etc.). Examples of the distance may include 0.01 mm, 0.1 mm, 0.5 mm, 1 mm, 2 mm, 5 mm, etc. The object may then be moved from a back of the build volume (or volume subset) frontward (e.g., along a y axis) until contacting with (or colliding with or coming within a distance from) another object or contacting with (or colliding with or coming within a distance from) a build volume (or volume subset) frontier. The object may then be moved from a right side of the build volume (or volume subset) leftward (e.g., along an x axis) until contacting with (or colliding with or coming within a distance from) another object or contacting with (or colliding with or coming within a distance from) a build volume (or volume subset) frontier. Other placement procedures may be utilized in some examples. For instance, other placement procedures that place the objects of the chromosome(s) proceeding in different directions may be utilized in some examples.

In performing selection, the apparatus may eliminate a quantity of the chromosomes. For instance, the apparatus may eliminate (e.g., discard, delete, remove, exclude, etc.) a quantity of the lowest ranked chromosomes (e.g., a percentage of lowest ranked chromosomes, a quantity of lowest ranked chromosomes, etc.). In some examples, the chromosomes may be categorized based on rank. For example, a first quantity (e.g., percentage of chromosomes, quantity of chromosomes, etc.) of the chromosomes may be categorized in a first category. For instance, the first quantity may be a set of highest ranked chromosomes. The first quantity of chromosomes may be referred to as elite chromosomes (e.g., a quantity of chromosomes with best fitness scores, quantities of objects packed, packing height, and/or packing density). In some examples, a gene or genes (e.g., object identifier(s) and/or pose(s)) of the elite chromosomes may be preserved and/or propagated to a subsequent (e.g., next) generation of chromosomes. A generation is a set of chromosomes (e.g., packings). For example, a generation may correspond to each iteration of the genetic procedure.

In some examples, selection may include chromosome selection for propagation (e.g., mutation and/or crossover) and/or packing (e.g., selecting a chromosome for a packing solution). For instance, the apparatus may select a chromosome or chromosomes based on a rank that is based on fitness scores, quantities of objects packed, packing density, and/or packing height, etc.

In some examples, selecting a chromosome may be based on a value or values. In some examples, selecting a chromosome for propagation is based on a fitness score. For example, a chromosome or chromosomes with better (e.g., a set of top) fitness scores may be selected for propagation and/or packing. For instance, selecting a chromosome for propagation may be based on a quantity of objects packed. For instance, chromosomes with higher quantities of objects packed, higher packing density, and/or lower packing height may be selected for propagation and/or packing. In some examples, first portions of chromosomes corresponding to the first object(s) may be propagated without modification to a subsequent generation or generations. In some examples, the chromosomes may not include portions corresponding to the first object(s).

In performing crossover, the apparatus may utilize a second quantity of chromosomes and elite chromosomes. The second quantity of the chromosomes may be referred to as crossover chromosomes. The second quantity of chromosomes may be ranked below the elite chromosomes. For instance, the apparatus may randomly select chromosomes that are ranked below the elite chromosomes to select the crossover chromosomes. The apparatus may crossover the crossover chromosomes with the elite chromosomes. For example, the apparatus may combine a part or parts (e.g., gene(s), object identifier(s), pose(s), location(s), orientation(s), rotation(s), and/or sequence position(s)) of an elite chromosome with a part or parts (e.g., gene(s), object identifier(s), pose(s), location(s), orientation(s), rotation(s), and/or sequence position(s)) of a crossover chromosome to generate a child chromosome in a subsequent (e.g., next) generation of chromosomes.

In performing mutation, the apparatus may randomly mutate an elite chromosome or elite chromosomes. For example, the apparatus may randomly change a gene or genes (e.g., object identifier(s), pose(s), location(s), orientation(s), rotation(s), and/or sequence position(s)) of an elite chromosome or elite chromosomes. The chromosomes to be mutated may be referred to as mutation chromosomes.

In some examples, a constant population size (e.g., quantity of chromosomes) may be maintained over generations. In some examples, the population size may vary over generations. In some examples, new random chromosomes may be added. For instance, a set of randomly generated chromosomes may be added to the elite chromosomes, crossover chromosomes, and mutation chromosomes. For instance, adding new random chromosomes may maintain the constant population size. The random chromosomes may be useful for driving a random search in different directions in the solution space. In some approaches, mutations of chromosomes may be represented as random movements in the object sequence and/or random rotations on the object orientations. The mutation stage may be utilized to mutate elite chromosomes (e.g., good solutions) into chromosomes with increased fitness (e.g., better solutions). Performing selection, crossover, and/or mutation may produce a subsequent generation of chromosomes.

In some examples, an operation or operations of the genetic procedure may repeat (e.g., iterate, recur, etc.). For example, evaluation, crossover, and/or mutation may be repeated for subsequent generations of chromosomes until an end condition is met (e.g., a threshold quantity of generations, a threshold quantity of packed objects, a threshold fitness score, and/or a threshold packing score, etc.).

The apparatus may determine 104, using a genetic procedure in accordance with the first priority and the second priority, a first arrangement within the first group and a second arrangement within the second group to produce a packing in a volume. An arrangement is an organization of objects and/or layout of objects. For example, an arrangement within a group may be a sequence of objects within the group and/or poses of the objects within the group. In some examples, an arrangement within a group may be a result of a genetic procedure operation or operations (e.g., initialization, mutation, evaluation, and/or selection) within the group.

In some examples, performing a genetic procedure in accordance with priorities (e.g., in accordance with a first priority, a second priority, etc.) may include performing a genetic procedure and/or an operation or operations of a genetic procedure in the priority order. For instance, a genetic procedure and/or an operation or operations of a genetic procedure may be performed in order of group priority (e.g., for the first group first, then for the second group, etc.). In some examples, the genetic procedure and/or an operation or operations of a genetic procedure may be performed in group priority order within an iteration (e.g., intra-iteration order). For instance, the genetic procedure and/or an operation or operations of a genetic procedure may be performed for a first group and then for a second group within an iteration. In an example, mutation may be performed within the first group first, and then may be performed within the second group. In some examples, the genetic procedure and/or an operation or operations of a genetic procedure may be performed for a first group for an amount of iterations, and then may be performed for a second group for an amount of iterations (e.g., inter-iteration order).

In some examples, mutations may be applied within groups (e.g., may be limited to application within each group). For instance, the method 100 (e.g., using a genetic procedure) may include applying mutations within the first group and within the second group. In some examples, the mutations may include reordering objects within the first group and within the second group. For instance, performing mutation may include changing the sequence of objects within a group. For example, objects may be reordered separately within groups (and may not be reordered between groups, for instance).

In some examples, the mutations may include modifying orientations within the first group and within the second group. For instance, performing mutation may include changing the orientations of objects within a group. For example, object orientations may be modified separately within groups.

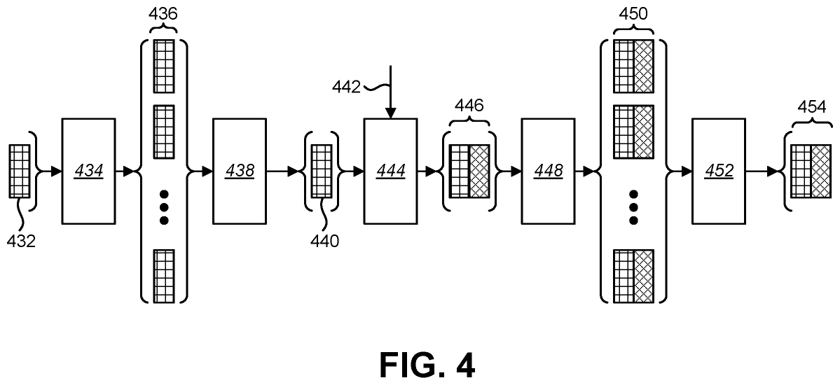

In some examples, using the genetic procedure in accordance with the first priority and the second priority may include performing a first genetic procedure instance for the first priority and a second genetic procedure instance for the second priority. A genetic procedure instance is the performance of a genetic procedure to an end condition (e.g., multiple iterations are performed until an end condition is reached) for an object group and/or chromosome. For example, a genetic procedure instance for a first group with a first priority may be performed until an end condition is reached for the first group. A genetic procedure instance for a second group with a second priority may then be performed until an end condition is reached for the second group (and/or a combination of the first group and the second group, for example). In some examples, using the genetic procedure may include performing the first genetic procedure instance on the first group to determine the first arrangement, concatenating the second group with the first arrangement, and performing the second genetic procedure instance on the second group to determine the second arrangement. For example, performing the first genetic procedure instance on the first group may produce a set of first group variations. The first arrangement may be selected from the set of first group variations. The second group may be concatenated with (e.g., added in order after) the first arrangement. The second genetic procedure instance may be performed on the second group (and/or on a combination of the first arrangement and the second group) to produce a set of second group variations. The second arrangement may be selected from the set of second group variations. In some examples, the first arrangement may be static during the second genetic procedure instance. In some examples, the first arrangement may be mutated during the second genetic procedure instance. An example of multiple genetic procedure instances is given in relation to FIG. 4.

In some examples, the method 100 may include selecting a packing based on the chromosomes (e.g., chromosomes after a generation or generations). For example, the method 100 may include selecting a packing based on a fitness score (e.g., ranking, value(s), packing score, packing density, packing height, etc.). In some examples, the method 100 may include determining the fitness score based on a placement procedure. For example, the apparatus may use the placement procedure described above to evaluate a fitness score for each chromosome resulting from the genetic procedure. In some examples, selecting the packing is based on the genetic procedure with an objective. For instance, the apparatus may select a chromosome that represents a packing with a highest ranking, best value(s), best packing score, maximum quantity of packed objects, best fitness score, maximum packing density, and/or minimum height, etc. In some examples, the apparatus may select the packing based on an objective (e.g., packing density, quantity of packed objects, packing height, and/or fitness, etc.) or a combination of objectives. For instance, a packing with a best combination of objectives, such as quantity of packed objects, packing density, packing height, and/or fitness score, may be selected.

In some examples, the packing may be executed to manufacture the objects. For example, the objects may be manufactured by an apparatus (e.g., 3D printer) in accordance with the selected packing. For instance, an apparatus may send the packing to another device (e.g., 3D printer) or may execute the packing to manufacture the objects in the packing. Some examples of the techniques described herein may be utilized in a variety of additive manufacturing. Some additive manufacturing techniques may be powder-based and driven by powder fusion. Some additive manufacturing techniques may include metal printing, such as metal jet fusion. Some examples of the approaches described herein may be utilized in powder bed fusion-based additive manufacturing, such as Selective Laser Melting (SLM), Selective Laser Sintering (SLS), Multi-Jet Fusion (MJF), etc.

In some examples, the method 100 may include presenting the selected packing. For instance, the apparatus may send an image of the packing to a display for presentation. In some examples, the method 100 may include sending the selected packing to another device (e.g., computing device, server, etc.). For instance, the apparatus may transmit the selected packing to another device via a network (e.g., a local area network, the Internet, etc.).

FIG. 2 is a block diagram of an example of an apparatus 202 that may be used in object group packing. The apparatus 202 may be an electronic device, such as a personal computer, a server computer, a printer, a 3D printer, a smartphone, a tablet computer, etc. The apparatus 202 may include and/or may be coupled to a processor 204 and/or a memory 206. In some examples, the apparatus 202 may be in communication with (e.g., coupled to, have a communication link with) an additive manufacturing device (e.g., a 3D printing device). In some examples, the apparatus 202 may be an example of a 3D printing device. The apparatus 202 may include additional components (not shown) and/or some of the components described herein may be removed and/or modified without departing from the scope of the techniques described herein.

The processor 204 may be any of a central processing unit (CPU), a semiconductor-based microprocessor, graphics processing unit (GPU), field-programmable gate array (FPGA), an application-specific integrated circuit (ASIC), and/or other hardware device suitable for retrieval and execution of instructions stored in the memory 206. The processor 204 may fetch, decode, and/or execute instructions (e.g., chromosome organization instructions 210, genetic procedure instructions 214, and/or packing selection instructions 216) stored in the memory 206. In some examples, the processor 204 may include an electronic circuit or circuits that include electronic components for performing a function or functions of the instructions (e.g., chromosome organization instructions 210, genetic procedure instructions 214, and/or packing selection instructions 216). In some examples, the processor 204 may perform one, some, or all of the functions, operations, elements, methods, etc., described in relation to one, some, or all of FIGS. 1-4.

The memory 206 may be any electronic, magnetic, optical, or other physical storage device that contains or stores electronic information (e.g., executable instructions, executable code, and/or data). The memory 206 may be, for example, Random Access Memory (RAM), Electrically Erasable Programmable Read-Only Memory (EEPROM), a storage device, an optical disc, and the like. In some examples, the memory 206 may be volatile and/or non-volatile memory, such as Dynamic Random Access Memory (DRAM), EEPROM, magnetoresistive random-access memory (MRAM), phase change RAM (PCRAM), memristor, flash memory, and the like. In some examples, the memory 206 may be a non-transitory tangible machine-readable (e.g., computer-readable) storage medium, where the term “non-transitory” does not encompass transitory propagating signals. In some examples, the memory 206 may include multiple devices (e.g., a RAM card and a solid-state drive (SSD)).

In some examples, the apparatus 202 may include an input/output interface (not shown in FIG. 2) through which the processor 204 may communicate with an external device or devices (not shown), for instance, to receive and store information (e.g., object data 208) pertaining to an object or objects to be manufactured (e.g., printed). The input/output interface may include hardware and/or machine-readable instructions to enable the processor 204 to communicate with the external device or devices. The input/output interface may enable a wired and/or wireless connection(s) to the external device or devices. The input/output interface may further include a network interface card and/or may also include hardware and/or machine-readable instructions to enable the processor 204 to communicate with various input and/or output devices, such as a keyboard, a mouse, a display, another apparatus, electronic device, computing device, etc., through which a user may input instructions into the apparatus 202.

In some examples, the memory 206 may store object data 208. The object data 208 may be obtained (e.g., received) from an external device and/or may be generated on the apparatus 202. For example, the processor 204 may execute instructions (not shown in FIG. 2) to receive the object data 208 from an external device.

The object data 208 may include data indicating objects and/or shapes (e.g., 3D models, dimensions, etc.) of objects. For example, the object data 208 may indicate objects for packing and/or manufacture.

In some examples, the processor 204 may execute chromosome organization instructions 210 to organize objects in a chromosome into a plurality of groups, where the plurality of groups is organized in a priority order based on a sorting factor. In some examples, organizing the objects in a chromosome into a plurality of groups may be performed as described in relation to FIG. 1. For instance, the processor 204 may sort the objects into groups (e.g., two groups, three groups, four groups, etc.) based on the sorting factor(s) associated with the objects and/or determined for the objects.

In some examples, the processor 204 may execute the chromosome organization instructions 210 to initialize a population of chromosomes from the organized chromosome. For example, the processor 204 may randomize objects (e.g., object order and/or orientations) within each group to determine variations of the chromosomes in the initial population. In some examples, initializing the chromosome(s) may be performed as described in relation to FIG. 1.

In some examples, the processor 204 may execute the genetic procedure instructions 214 to execute a genetic procedure on the plurality of groups in a priority order to determine a packing. In some examples, executing the genetic procedure may be performed as described in relation to FIG. 1. For instance, the processor 204 may perform mutation, evaluation, and/or selection on the plurality of groups in the priority order (e.g., intra-iteration order, inter-iteration order). In some examples, the processor 204 may execute the genetic procedure to perform intra-group mutation in the priority order. For instance, mutation may be performed separately within each group in the priority order (e.g., from highest priority group to lowest priority group).

In some examples, the processor 204 may execute the genetic procedure recursively with instances for the plurality of groups. For example, the processor 204 may perform a genetic procedure instance for each group in the priority order. In some examples, multiple high-ranked local arrangements may be utilized as building blocks for a global packing. In some examples, using the genetic procedure recursively and/or using multiple instances may provide independent processing and/or increased coverage of a search space.

In some examples, evaluation may be performed based on a group, groups, and/or whole chromosomes. For example, the processor 204 may rank the chromosomes based on a group (e.g., based on the first group for a first iteration or instance, then based on a second group for a second iteration or instance, etc.), groups (e.g., based on a first group for a first instance, then based on a first group and second group for a second instance, etc.), or whole chromosomes. For instance, the processor 204 may rank the chromosomes based on values, fitness scores, quantities of packed objects, packing densities, and/or packing heights, etc., determined for a group, groups, and/or chromosome.

In some examples, the processor 204 may execute the genetic procedure instructions 214 to propagate a chromosome or chromosomes in a case that the evaluation is satisfied. In some examples, propagating the chromosome(s) in a case that the evaluation is satisfied may be performed as described in relation to FIG. 1. For example, the processor 204 may propagate (e.g., mutate and/or crossover) a group or groups of a chromosome or chromosomes that are above a ranking threshold. The apparatus 202 (e.g., processor 204) may iterate evaluating and propagating chromosomes for a generation or generations in some approaches.

In some examples, the processor 204 may execute the packing selection instructions 216 to select a packing based on the chromosome(s). For example, the processor 204 may select a chromosome as a packing with a maximum quantity of packed objects, best fitness score, highest packing density, smallest packing height, and/or best packing score from the chromosomes (e.g., packings based on the chromosomes). In some examples, the apparatus 202 may perform manufacturing based on the selected packing. For example, the apparatus 202 may 3D print the selected packing and/or send the selected packing to a 3D printer for manufacturing.

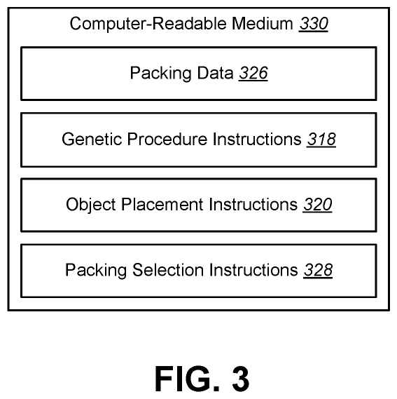

FIG. 3 is a block diagram illustrating an example of a computer-readable medium 330 for performing object group packing. The computer-readable medium is a non-transitory, tangible computer-readable medium 330. The computer-readable medium 330 may be, for example, RAM, EEPROM, a storage device, an optical disc, and the like. In some examples, the computer-readable medium 330 may be volatile and/or non-volatile memory, such as DRAM, EEPROM, MRAM, PCRAM, memristor, flash memory, and the like. In some examples, the memory 206 described in relation to FIG. 2 may be an example of the computer-readable medium 330 described in relation to FIG. 3.

The computer-readable medium 330 may include code (e.g., data and/or instructions). For example, the computer-readable medium 330 may include packing data 326, genetic procedure instructions 318, object placement instructions 320, and/or packing selection instructions 328.

The packing data 326 may include information indicating a plurality of packings. For example, the packing data 326 may indicate a set of packings, a population of packings, and/or generations of packings. For instance, operations may be performed as described herein to determine a plurality of chromosomes that represent packings. The chromosomes may be stored as packing data 326.

In some examples, the genetic procedure instructions 318 are code to cause a processor to execute a genetic procedure on a chromosome that includes prioritized groups of objects to produce mutated chromosomes. In some examples, executing the genetic procedure on a chromosome that includes prioritized groups of objects to produce mutated chromosomes may be performed as described in relation to FIG. 1 and/or FIG. 2. In some examples, mutations of the prioritized groups are mutually exclusive. For instance, the processor may perform mutations (e.g., random reordering, random orientation modification, and/or crossover, etc.) within each of the prioritized groups.

In some examples, the object placement instructions 320 are code to cause the processor to execute a placement procedure to determine the fitness scores. In some examples, performing a placement procedure may be accomplished as described in relation to FIG. 1. For instance, the object placement instructions 320 may include code to cause the processor to use a placement procedure (e.g., bottom-front-left placement procedure, etc.) to place the objects of the chromosomes. The placed objects may be utilized to determine the fitness scores. For instance, the processor may determine quantity of objects packed, packing density, packing height, and/or packing score, etc., for each of the mutated chromosomes.

In some examples, the packing selection instructions 328 are code to cause the processor to select a packing from the mutated chromosomes based on fitness scores corresponding to the mutated chromosomes. For instance, the packing may be selected from the mutated chromosomes (e.g., placed objects of the mutated chromosomes) based on values, fitness scores, quantities of objects in the placements (e.g., packings), object densities, and/or heights of the placements (e.g., packings). In some examples, the packing selection instructions 328 are code to cause the processor to determine values, fitness scores, quantities of objects in the placements (e.g., packings), object densities, and/or heights of the placements (e.g., packings) of chromosomes. For instance, the processor may execute the genetic procedure instructions 318 to determine, look-up, count, etc., the quantities of objects of the placements. In some examples, the processor may determine a density and/or height (e.g., z-dimension) for placements (e.g., packings) indicated by the chromosomes. For instance, the fitness score may be a height of the placement (e.g., chromosome) and/or a density of the placement (e.g., chromosome). In some examples, determining the fitness score of the placement (e.g., chromosome) may be performed as described in relation to FIG. 1 and/or FIG. 2.

In some examples, the packing selection instructions 328 are code to cause the processor to determine a packing based on the values, fitness scores, quantities of objects in the placements (e.g., packings), object densities, and/or heights of the placements (e.g., packings). For instance, the packing selection instructions 328 may cause a processor to determine a best packing in terms of values, fitness scores, quantities of objects in the placements (e.g., packings), object densities, and/or heights of the placements (e.g., packings) from a set (e.g., generation or generations) of chromosomes. In some examples, determining the packing may be performed as described in relation to FIG. 1 and/or FIG. 2.

In some examples, the packing selection instructions 328 may include code to cause the processor to rank chromosomes with hierarchical criteria based on values, fitness scores, quantities of objects in the placements (e.g., packings), object densities, and/or heights of the placements (e.g., packings). The hierarchical criteria may include criteria with varying priority. For instance, the quantity of objects may be a criterion at a top hierarchical level and the fitness score may be a criterion at a next lower hierarchical level. In the hierarchical criteria, the processor may determine if there is a chromosome (e.g., placement, packing) with a best top hierarchical level criterion. If there is more than one chromosome with a best top hierarchical level criterion (e.g., multiple chromosomes with the same maximum quantity of packed objects), the selection may utilize the next lower criterion. If there is more than one chromosome with a best next lower criterion (e.g., multiple chromosomes with the same fitness scores or fitness scores within a range), the selection may utilize the next lower hierarchical level criterion (e.g., lowest level).

To determine a packing in some examples, packing scores may be compared. A packing score is a score indicating a value or a combination of values (e.g., quantity of objects packed, fitness score, packing height, and/or packing density, etc.). An example of packing selection is given as follows. Assume two packings A and B with corresponding packing scores s_A and s_B. In some examples, a packing score s_X may include parameters (e.g., k parameters or values): s_X={p1_X, p2_X, . . . , pk_X}, where px_X is a parameter or value. A quantity (e.g., k) of threshold values may be utilized: α, β, . . . , φ. If the first k−1 parameters of each packing are relatively closer to each other (up to an adjustable threshold), a Boolean value of (p1_A>p1_B) may indicate whether s_A is greater than s_B. For instance, In a case that abs(p1_A−p1_B)<α and abs(p2_A−p2_B)<β and so on for the rest of the parameters, and (pk_A !=pk_B), then a Boolean value of (pk_A>pk_B) may indicate whether s_A is greater than s_B. Otherwise, a parameter number k−1 may be compared. For instance, in a case that abs(p1_A−p1_B)<α and abs(p2_A−p2_B)<β and so on for the rest of the parameters, and (pk−1_A !=pk−1_β), then a Boolean value of (pk−1_A>pk−1_β) may indicate whether s_A is greater than s_B. Otherwise, similar comparisons may be made for a parameter or parameters (e.g., pk−2_A !=pk−2_B and so on) until the final case, where (p1_A>p1_B) may indicate whether s_A is greater than s_B. The packing with the greatest score may be selected. In some examples, one parameter or multiple parameters (e.g., values) may be utilized in the comparison.

In some examples, the computer-readable medium 330 may include code for causing a processor to manufacture the selected packing. For example, the code may cause a processor to execute instructions and/or send instructions to another device (e.g., 3D printer) to manufacture the selected packing.

FIG. 4 is a diagram illustrating an example of recursive genetic procedures in accordance with some examples of the techniques described herein. In some examples, the method 100 may include an aspect or aspects of the recursive genetic procedures, the apparatus 202 may perform an aspect or aspects of the recursive genetic procedures, and/or instructions for an aspect or aspects of the recursive genetic procedures may be stored in the computer-readable medium 330. In some examples, each object group with a corresponding priority may be processed by applying a respective (e.g., independent) genetic procedure instance. The object groups may preserve their corresponding objects through the genetic procedure instances. In some examples, each group may be processed sequentially in priority order.

In the example of FIG. 4, a first group 432 with a first (e.g., highest) priority may be utilized in a first genetic procedure instance 434. The first genetic procedure instance 434 may perform initialization, mutation, crossover, evaluation, and/or selection operations on the first group 432 to produce a set of first arrangements 436 (e.g., mutated first groups). The first arrangements 436 may be provided to a first election procedure 438. The first election procedure 438 may produce an elected first arrangement 440 from the set of first arrangements 436.

In the example of FIG. 4, one elected first arrangement 440 is shown. In some examples, a quantity of (e.g., k) first arrangements may be elected for combination with another group or groups. For instance, k first arrangements may be elected randomly from the set of first arrangements (e.g., last generation of first arrangements) or by taking the k top-ranked first arrangements.

In the example of FIG. 4, the first group 432 is provided to a first genetic procedure instance 434, where a quantity of random first arrangements may be created and processed (e.g., mutated and/or optimized). In the example of FIG. 4, the first election procedure 438 is utilized to produce a (k=1 highest ranked) elected first arrangement 440.

For each elected first arrangement, a second genetic procedure instance may be performed. For instance, a second genetic procedure instance may utilize a second group with the objects (e.g., 0-ith objects) from a first genetic procedure instance. In the example of FIG. 4, the elected first arrangement 440 is provided to a concatenation procedure 444. The concatenation procedure 444 may concatenate the elected first arrangement 440 with a second group 442 to produce concatenated groups 446 that include the elected first arrangement 440 and the second group 442.

In the example of FIG. 4, the concatenated groups 446 may be utilized in a second genetic procedure instance 448. The second genetic procedure instance 448 may perform initialization, mutation, crossover, evaluation, and/or selection operations on the concatenated groups 446 to produce a set of second arrangements 450 (e.g., first arrangements with mutated second groups). The second arrangements 450 may be provided to a second election procedure 452. The second election procedure 452 may produce an elected second arrangement 454 from the set of second arrangements 450. In some examples, further genetic procedure instances and/or groups may be utilized.

In some examples, after each genetic procedure instance, a number (e.g., constant number) of arrangements may be selected as input for a further genetic procedure instance or instances. The quantity of group and/or arrangement combinations may grow exponentially over genetic procedure instances.

After the last genetic procedure instance (e.g., processing of the last group), all of the chromosomes may be ranked according to fitness score. The highest ranked chromosome may be selected as the packing (e.g., global packing solution).

Some examples of the techniques described herein may enable automated prioritized build bed packing with genetic procedure-based packing. Some examples of the techniques described may use chromosome manipulation to achieve near-optimal packing density while giving packing priority to high-priority objects or a group of objects. For instance, a chromosome may be subdivided to handle packing objects following a priority or rank. For example, in a digital manufacturing environment, service level agreements (e.g., pricing, delivery time, throughput, etc.) may drive job priorities, where some jobs may be more urgent than others. In some examples, priority may be determined based on received input and/or determined automatically based on a function (e.g., function of a sorting factor). In some examples, objects may be split into groups and/or categories, where each group is processed and/or packed independently. A global packing may be achieved by concatenating group packings and/or group arrangements.

As used herein, the term “and/or” may mean an item or items. For example, the phrase “A, B, and/or C” may mean any of: A (without B and C), B (without A and C), C (without A and B), A and B (but not C), B and C (but not A), A and C (but not B), or all of A, B, and C.

While various examples are described herein, the disclosure is not limited to the examples. Variations of the examples described herein may be implemented within the scope of the disclosure. For example, aspects or elements of the examples described herein may be omitted or combined.

1. A method, comprising:
ordering objects of a chromosome into a first group and a second group in accordance with a first priority and a second priority; and
determining, using a genetic procedure in accordance with the first priority and the second priority, a first arrangement within the first group and a second arrangement within the second group to produce a packing in a volume.

2. The method of claim 1, wherein ordering the objects comprises sorting the objects based on a sorting factor.

3. The method of claim 1, comprising applying mutations within the first group and within the second group.

4. The method of claim 3, wherein the mutations comprise reordering objects within the first group and within the second group.

5. The method of claim 3, wherein the mutations comprise modifying orientations within the first group and within the second group.

6. The method of claim 1, wherein using the genetic procedure in accordance with the first priority and the second priority comprises performing a first genetic procedure instance for the first priority and a second genetic procedure instance for the second priority.

7. The method of claim 6, wherein using the genetic procedure comprises:
performing the first genetic procedure instance on the first group to determine the first arrangement;
concatenating the second group with the first arrangement; and
performing the second genetic procedure instance on the second group to determine the second arrangement.

8. The method of claim 1, comprising selecting the packing based on a fitness score.

9. The method of claim 8, comprising determining the fitness score based on a placement procedure.

10. An apparatus, comprising:
a memory;
a processor coupled to the memory, wherein the processor is to:
organize objects in a chromosome into a plurality of groups, wherein the plurality of groups is organized in a priority order based on a sorting factor; and
execute a genetic procedure on the plurality of groups in the priority order to determine a packing.

11. The apparatus of claim 10, wherein the processor is to execute the genetic procedure to perform intra-group mutation in the priority order.

12. The apparatus of claim 10, wherein the processor is to execute the genetic procedure recursively with instances for the plurality of groups.

13. A non-transitory tangible computer-readable medium storing executable code, comprising:
code to cause a processor to execute a genetic procedure on a chromosome that includes prioritized groups of objects to produce mutated chromosomes; and
code to cause the processor to select a packing from the mutated chromosomes based on fitness scores corresponding to the mutated chromosomes.

14. The computer-readable medium of claim 13, further comprising code to cause the processor to execute a placement procedure to determine the fitness scores.

15. The computer-readable medium of claim 13, wherein mutations of the prioritized groups are mutually exclusive.
